(19) United States
(12) Patent Application Publication
(10) Pub. No.: US 2008/0051388 A1
(43) Pub. Date: Feb. 28, 2008

(54) Novel Compounds That Inhibit Factor Xa Activity

(75) Inventors: Thilo Fuchs, Neuried (DE); Robert Eckl, Munchen (DE)

Correspondence Address: EDWARDS ANGELL PALMER & DODGE LLP, P.O. BOX 55874, BOSTON, MA, 02205, US

(73) Assignee: Morphochem Aktiengesellschaft Fur Kombinatorische Chemie, Munchen (DE)

(21) Appl. No.: 11/632,291

(22) PCT Filed: Jul. 19, 2005
(86) PCT No.: PCT/EP05/07867

§ 371 Date: Jan. 11, 2007

(30) Foreign Application Priority Data
Jul. 19, 2004 (DE) 10 2004 034 913.4

(51) Int. Cl. A61K 31/445; A61K 31/541; A61K 31/55; A61P 7/02; C07D 243/08; C07D 405/02; C07D 417/02

(52) U.S. Cl. 514/218; 514/227.8; 514/326; 540/575; 544/374; 544/60

(57) ABSTRACT

The present invention relates to compounds of formula (I): 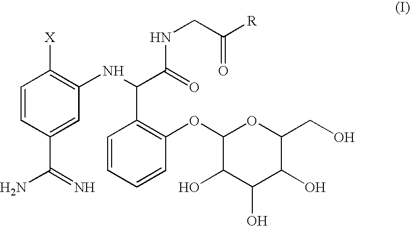 or pharmaceutically acceptable salts, solvates, hydrates or pharmaceutically acceptable formulations thereof. Those compounds can be used in inhibiting factor Xa and in the prevention and/or treatment of thrombolytic conditions.

The present invention relates to new compounds having an inhibitory action on blood clotting (so-called anticoagulants) and to their pharmacologically acceptable salts and solvates and hydrates, to pharmaceutical compositions comprising them as active ingredient, to processes for the preparation of such compounds, salts and compositions, and to the use thereof in the prevention and/or treatment of thromboembolic conditions. Those compounds, salts and compositions are very effective factor Xa inhibitors. The present invention relates also to pro-drugs, optically active forms, racemates and diastereoisomers of those compounds and salts.

Thromboembolic conditions are caused by an increased tendency to blood clotting in people with risk factors such as, for example, relatively major operations, prolonged immobilisation, fractures of the lower extremities, obesity, blood fat metabolism disorders, infections with gram-negative organisms, cancer and older age.

Venous thromboses may lead to the development of oedema or inflammation of the tissue drained by the affected vein. Thrombosis of a deeper vein (so-called deep vein thrombosis) may lead to serious complications, such as, for example, pulmonary embolism. Arterial thrombosis may lead to ischaemic necrosis of the tissue supplied by the affected artery, such as, for example, to myocardial infarct in the case of an affected coronary artery. Other thromboembolic conditions are, for example, arterio-sclerosis, apoplexy (stroke), angina pectoris, intermittent claudication.

Under normal physiological conditions, natural blood clotting protects against major blood loss from a damaged blood vessel. During blood clotting, liquid blood is converted into a blood clot, a gelatinous mass which seals injured blood vessels by forming a plug. In that process, soluble fibrinogen present in the plasma is converted into the fibrous-gelatinous clotting substance fibrin in a multi-stage process, the so-called coagulation cascade.

A distinction is made between two different pathways of coagulation activation. The intrinsic coagulation pathway is initiated when blood comes into contact with non-physiological surfaces. The extrinsic coagulation pathway is initiated by injury to blood vessels. Both coagulation pathways join in a common pathway in which the coagulation factor X, a serine protease, is converted into its active form (factor Xa). Factor Xa, together with factor Va and Ca²⁺ in the so-called prothrombinase complex, causes prothrombin to be converted into thrombin which in turn, by cleaving peptides from fibrinogen, releases fibrin monomers, which are capable of coagulating to form fibrin fibres. Finally, factor XIII brings about cross-linking and thus stabilisation of the fibrin fibres.

Anticoagulants are used both for the prevention and for the treatment of thromboembolic conditions. As far as anticoagulants in the narrower sense are concerned, a distinction is made between heparin, which is immediately effective and which directly inhibits certain blood clotting factors, and vitamin K antagonists (for example, coumarin derivatives). The latter inhibit the production in the liver of certain clotting factors which is dependent on the presence of vitamin K, and begin to take effect only slowly. Other anticoagulant agents are the fibrinolytics, which bring about direct or indirect activation of the fibrinolytic system, and thrombocyte aggregation inhibitors, such as, for example, acetylsalicylic acid. A more seldom used method is reduction of the fibrinogen level in the blood by the enzyme ancrod. The object of using anticoagulant agents is to prevent the development of a blood clot that could close a vessel or also to dissolve it again once it has formed.

The above-mentioned anticoagulants in the narrower sense, that is to say heparin and vitamin K antagonists, have disadvantages. In the case of heparin, a distinction is made between unfractionated heparin (UFH) and low-molecular-weight heparin (LMWH). A disadvantage with UFH is the fact that it generally has to be administered intravenously, has a varying anticoagulant effect and therefore necessitates frequent monitoring of the patient and adaptation of the dosage. Although LMWH can be used subcutaneously in a constant, unmonitored dosage, its effect, compared to that of UFH, is greatly reduced because of its short chain length.

The vitamin K antagonists such as, for example, warfarin exhibit degrees of activity that differ from patient to patient—presumably owing to genetic factors. In addition to the slow onset of action mentioned above, this is associated with the disadvantage that patients have to be monitored and individual adaptation of the dosage is required.

Other known anticoagulants belong to the group of the thrombin inhibitors. Current overviews of relevant research activity in that field can be found, for example, in Jules A. Shafer, Current Opinion in Chemical Biology, 1988, 2: 458-485, Joseph P. Vacca, Current Opinion in Chemical Biology, 2000, 4: 394-400 and also in Fahad Al-Obeidi and James A. Ostrem, DDT, Vol. 3, No. 5, May 1998: 223-231.

A crucial disadvantage of thrombin inhibitors is that, in order to obtain the desired effect, it is necessary to suppress thrombin activity in vivo to such a great extent that the tendency to haemorrhage may increase, which makes dosage difficult.

In contrast, factor Xa inhibitors cause suppression of the new formation of thrombin from prothrombin, whereas they do not impair existing thrombin activity which is necessary for primary haemostasis.

An object of the present invention was to provide new compounds having useful properties, especially an anticoagulating action.

More precisely, the object was to provide new factor Xa inhibitors having improved efficacy, reduced side-effects and/or increased selectivity. In addition, suitable pharmaceutical compositions were to be provided. Those compounds and compositions were to be administrable preferably parenterally or orally, especially orally.

A further object of the present invention was to provide a process for the preparation of those new compounds.

Those new compounds were furthermore to be suitable for use in the prevention and/or treatment of thromboembolic conditions.

The present invention describes anticoagulant compounds, their pharmacologically acceptable salts and solvates and hydrates and formulations that have a high activity and selectivity and can be administered especially orally. The present invention further relates to pro-drugs, optically active forms, racemates and diastereoisomers of those compounds and salts. The said compounds and salts may also themselves be pro-drugs, which are activated only by metabolisation. Pharmaceutical compositions comprising the said compounds or salts etc. as active ingredient are also described.

The present invention relates to a compound of the general formula (I):

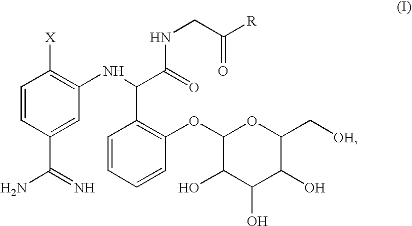

wherein

X is a hydrogen atom,a hydroxy group, a C₁—, C₂—, C₃— or C₄-alkyloxy group or a fluorine atom and

R is an optionally substituted heterocycloalkyl radical having 5, 6 or 7 ring atoms,

or a pharmacologically acceptable salt, solvate, hydrate or pharmacologically acceptable formulation thereof.

The following definitions relate to the entire description and, especially, to the claims:

The expression “alkyl” refers to a saturated, straight-chain or branched hydrocarbon group having, preferably, 1, 2, 3 or 4 carbon atoms, for example a methyl, ethyl, propyl, isopropyl, isobutyl, tert-butyl or n-butyl group.

Furthermore, the expression “alkyl” refers to groups in which one, two, three or more hydrogen atoms have been replaced by a halogen atom (preferably F or Cl) such as, for example, a 2,2,2-trichloroethyl or trifluoromethyl group.

The expression “heterocycloalkyl radical having 5, 6 or 7 ring atoms” refers to a cyclopentyl, cyclohexyl or cycloheptyl group in which one, two or three (preferably two) ring carbon atoms have been replaced, each independently of any other(s), by an oxygen, nitrogen or sulphur atom (preferably a nitrogen atom). The expression “heterocycloalkyl” refers furthermore to corresponding groups in which one or more hydrogen atoms have been replaced, each independently of any other(s), by fluorine, chlorine, bromine or iodine atoms or by OH, ═O, SH, ═S, NH₂, ═NH or NO₂ groups. Examples are a piperidyl, morpholinyl or piperazinyl group.

The expression “optionally substituted” refers to groups in which one, two or more hydrogen atoms have been replaced by fluorine, chlorine, bromine or iodine atoms or by OH, ═O, SH, ═S, SO₂NH₂, NH₂, ═NH, —C(═NH)NH₂ or NO₂ groups. The expression refers furthermore to groups in which one, two or more hydrogen atoms have been replaced, each independently of any other(s), by C₁-C₄alkyl groups, it being possible for the C₁-C₄alkyl groups to be substituted by OH, ═O, SH, ═S, SO₂NH₂, NH₂, 50 NH, —C(═NH)NH₂ or NO₂ groups.

Preference is given to X being a hydrogen atom or a hydroxy group (especially a hydrogen atom).

Further preference is given to R being a group of formula:

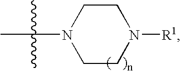

wherein n is 0, 1 or 2 (especially 1) and R¹ is a C₁—, C₂—, C₃— or C₄-alkyl group (especially a CH₃ or CF₃ group).

Special preference is given to R being a cyclic group of formula —N(CH₂CH₂)₂NCH₃ (an N-methylpiperazinyl or 4-methyl-piperazinyl group).

Further preference is given to the stereochemistry of the 3,4,5-trihydroxy-6-hydroxymethyl-tetrahydropyran-2-yloxy group corresponding to that of β-D-glucose.

Preference is also given to the following compound: 2-(3-carbamimidoyl-phenylamino)-N-[2-(4-methyl-piperazin-1-yl)-2-oxo-ethyl]-2-[2-(3,4,5-trihydroxy-6-hydroxymethyl-tetrahydro-pyran-2-yloxy)-phenyl]-acetamide; the stereo-chemistry of the 3,4,5-trihydroxy-6-hydroxymethyl-tetrahydropyran-2-yloxy group therein corresponds especially to that of β-D-glucose or of α-D-glucose, and further preference is given to the stereochemistry at the phenylglycine entity being of (S) configuration.

Special preference is given to the compound of formula (I) having one of the following structures:

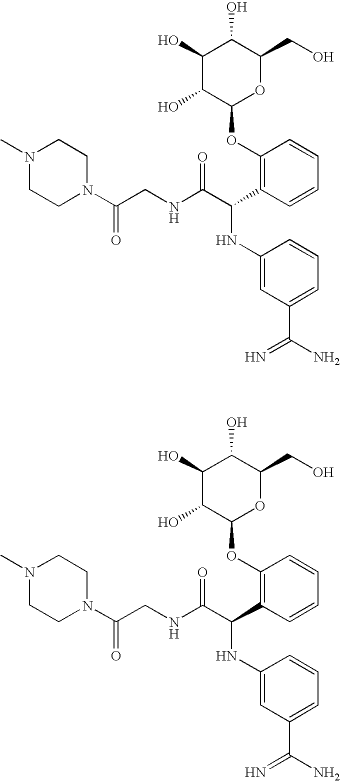

Owing to their substitution, compounds of formula (I) contain one or more centres of chirality. The present invention therefore includes both all pure enantiomers and all pure diastereoisomers and also mixtures thereof in any mixing ratio. The present invention moreover includes all tautomeric forms of the compounds of formula (I) or.

Examples of pharmacologically acceptable salts of compounds of formula (I) are salts of physiologically acceptable mineral acids, such as hydrochloric acid, sulphuric acid and phosphoric acid; or salts of organic acids, such as methanesulphonic acid, p-toluenesulphonic acid, lactic acid, formic acid, acetic acid, trifluoroacetic acid, citric acid, succinic acid, fumaric acid, maleic acid and salicylic acid.

Compounds of formula (I) can be solvated, especially hydrated. The hydration may take place, for example, during the preparation process or as a consequence of the hygroscopic nature of the initially anhydrous compounds of formula (I).

The pharmaceutical compositions according to the present invention comprise at least one compound of formula (I) as active ingredient and optionally carrier substances and/or adjuvants.

The pro-drugs (for example, R. B. Silverman, Medizinische Chemie, VCH Weinheim, 1995, Chapter 8, p. 361ff), to which the present invention also relates consist of a compound of formula (I) and at least one pharmacologically acceptable protecting group that is removed under physiological conditions, for example a hydroxy, alkoxy, aralkyloxy, acyl or acyloxy group, such as, for example, a methoxy, ethoxy, benzyloxy, acetyl or acetyloxy group; especially on the amidino group.

The compounds of formula (I) described herein can be prepared analogously to the methods described in WO0216312. In the case of the compounds obtained in that manner it was found that both the compounds of formula (I) having an (R) configuration at the phenylglycine entity and also the corresponding (S)-configured compounds are very effective factor Xa inhibitors, the (S)-configured compounds having, when identically substituted, slightly better inhibitory properties. Preference is therefore given in accordance with the invention to compounds of formula (I) having an (S) configuration at the phenylglycine entity, whilst compounds having an (R) configuration, and also mixtures in any mixing ratio, also have very good inhibitory properties and this invention relates also thereto.

3-Aminobenzamidine is commercially available; 3-amino-4-hydroxybenzamidine can be prepared from commercially available 4-hydroxy-3-nitrobenzonitrile by means of a Pinner reaction (A. Pinner, F. Klein, Ber. 10, 1889 (1877); 11, 4, 1475 (1878); 16, 352, 1643 (1883)) resulting in 4-hydroxy-3-nitro-benzamidine and subsequent reduction with H₂—Pd/C. Further benzamidines can also be prepared analogously.

Glycosylated aryl compounds (for example, glycosylated benzaldehydes) can be prepared, for example, by the processes described in Kleine et al. Carbohydrate Research 1985, 142, 333-337 and Brewster et al. Tetrahedron Letters 1979, 5051-5054; helicin (salicylaldehyde-β-D-glucoside) is commercially available.

All starting materials for which no synthesis is indicated are commercially available, for example from Acros, Aldrich, Fluka, Lancaster or Merck.

A compound or pharmaceutical composition of the present invention can be used in inhibiting factor Xa activity, in the prevention and/or treatment of thromboembolic conditions, arterial restenosis, septicaemia, cancer, acute inflammation or other conditions mediated by factor Xa activity, and especially venous thromboses, oedema or inflammation, deep vein thrombosis, pulmonary embolisms, thromboembolic complications after relatively major operations, in the case of vascular surgery, prolonged immobilisation, fractures of the lower extremities etc., arterial thromboses, especially of the coronary vessels in the event of myocardial infarct, and arteriosclerosis, stroke, angina pectoris, intermittent claudication, to mention but a few indications.

In general, as mentioned at the beginning, the active ingredients according to the invention are to have an inhibitory action towards factor Xa that is as great as possible while having a selectivity that is as high as possible. The selectivity was assessed in the present case by comparing the inhibitory action towards factor Xa and also tryptase, trypsin, plasmin, thrombin and further serine proteases. Furthermore, the present compounds according to the invention are of interest as inhibitors of further enzymes of the coagulation cascade (extrinsic and intrinsic) such as, for example, factor II, factor VII, factor VIIa, factor IX, factor IXa and factor X.

As mentioned above, the therapeutic use of the compounds of formula (I), of their pharmacologically acceptable salts and solvates and hydrates and also formulations and pharmaceutical compositions lies within the scope of the present invention.

The present invention relates also to the use of those active ingredients in the preparation of medicaments for the prevention and/or treatment of thromboembolic conditions. In general, compounds of formula (I) are administered either individually or in combination with any other desired therapeutic agent, using the known and acceptable methods. Administration may be effected, for example, by one of the following routes: orally, for example in the form of dragées, coated tablets, pills, semi-solid substances, soft or hard capsules, solutions, emulsions or suspensions; parenterally, for example in the form of an injectable solution; rectally in the form of suppositories; by inhalation, for example in the form of a powder formulation or spray, transdermally or intranasally. For the preparation of such tablets, pills, semi-solid substances, coated tablets, dragées and hard gelatin capsules, the therapeutically usable product can be mixed with pharmacologically inert, inorganic or organic pharmaceutical carrier substances, for example with lactose, sucrose, glucose, gelatin, malt, silica gel, starch or derivatives thereof, talcum, stearic acid or salts thereof, skimmed milk powder and the like. For the preparation of soft capsules, pharmaceutical carrier substances such as, for example, vegetable oils, petroleum, animal or synthetic oils, wax, fat and polyols can be used. For the preparation of liquid solutions and syrups, pharmaceutical carrier substances such as, for example, water, alcohols, aqueous saline solution, aqueous dextrose, polyols, glycerol, vegetable oils, petroleum and animal or synthetic oils can be used. For suppositories, pharmaceutical carrier substances such as, for example, vegetable oils, petroleum, animal or synthetic oils, wax, fat and polyols can be used. For aerosol formulations, compressed gases that are suitable for the purpose can be used, such as, for example, oxygen, nitrogen and carbon dioxide. The pharmaceutically acceptable agents may also comprise additives for preserving and stabilising, emulsifiers, sweeteners, flavourings, salts for altering the osmotic pressure, buffers, encapsulation additives and anti-oxidants.

Combinations with other therapeutic agents may comprise other active ingredients that are customarily used for the prevention and/or treatment of thromboembolic conditions, such as, for example, warfarin etc.

For the prevention and/or treatment of the conditions mentioned above, the dose of the biologically active compound according to the invention can vary within wide limits and can be adjusted to individual requirements. In general, a dose of from 0.1 μg to 10 mg/kg of body weight per day is suitable, a preferred dose being from 0.1 to 4 mg/kg per day. In suitable cases, the dose may also be below or above the stated values.

The daily dose can be administered in, for example, 1, 2, 3 or 4 individual doses. It is also possible to administer the dose as a single dose for, for example, one week.

The following Examples are intended to illustrate the invention. The stereochemistry of 3,4,5-trihydroxy-6-hydroxymethyl-tetrahydropyran-2-yloxy corresponds to that of β-D-glucose.

### EXAMPLES

### Example 1

### 2-(3-Carbamimidoyl-phenylamino)-N-[2-(4-methyl-piperazin-1-yl)-2-oxo-ethyl]-2-[2-(3,4,5-trihydroxy-6-hydroxymethyl-tetrahydro-pyran-2-yloxy)-phenyl]-acetamide

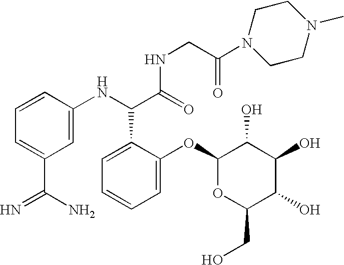

3-Aminobenzamidine dihydrochloride (6.243 g, 30 mmol) is weighed into a flask and, in parallel, helicin (8.528 g, 30 mmol), toluene-4-sulfonic acid monohydrate (5.707 g, 30 mmol) and 2-isocyano-1-(4-methyl-piperazin-1-yl)-ethanone (5.016 g, 30 mmol) are weighed into three glass beakers. Then 120 ml of acetonitrile:water (1:1) are added to the flask. Then, with stirring, the helicin and the toluene-4-sulfonic acid monohydrate are added spatula-wise at room temperature, one after the other, over a period of 30 minutes. In parallel thereto, 2-isocyano-l-(4-methyl-piperazin-1-yl)-ethanone is dissolved in 100 ml of acetonitrile:water (1:1) and transferred to a dropping funnel (rinsing with 20 ml of acetonitrile:water [1:1]). After the helicin, 3-aminobenzamidine dihydrochloride and toluene-4-sulfonic acid monohydrate have been stirred for 10 minutes at room temperature, the yellow solution is cooled to 0° C. and, with vigorous stirring, the 2-isocyano-1-(4-methyl-piperazin-1-yl)-ethanone solution is then added dropwise at 0° C. The dropwise addition procedure is complete after two hours. Stirring is continued for a further two hours at 0° C. (four hours in total). The solvent is then removed in vacuo. By means of HPLC, the residue is purified and the diastereoisomers are separated.
C₂₈H₃₈N₆O₈ (586.65)
MS (ESI): 587 [M+H]

General procedure for the preparation of the isocyanoacetamides used, from seconary amines (cf. K. Matsumoto et al., Synthesis, 1997, 249-250):

The secondary amine (1 mmol) is introduced into a single-necked flask either without solvent or in a solvent such as methanol, dichlormethane or dimethylformamide. Isocyano-acetic acid methyl ester (1 mmol) is speedily added dropwise by means of a dropping funnel. The solution is stirred at room temperature for 18 hours.

Either the solution is already fully crystallised after that time or crystallisation occurs after addition of ether and storage in a deep-freeze cabinet overnight. The solid formed is carefully broken up using a spatula and suspended in diethyl ether. The solid is then filtered off under suction and washed several times with diethyl ether.

In Example 1, N-methylpiperazine was used as the amine component for the isonitrile.

### Example 2

### 2-(3-Carbamimidoyl-phenylamino)-N-[2-(4-methyl-piperazin-1-yl)-2-oxo-ethyl]-2-[2-(3,4,5-trihydroxy-6-hydroxymethyl-tetrahydro-pyran-2-yloxy)-phenyl]-acetamide

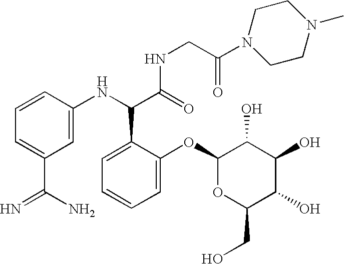

See Example 1 for synthesis.
C₂₈H₃₈N₆O₈ (586.65)
MS (ESI): 587 [M+H]

### Example 3

### 2-(5-Carbamimidoyl-2-hydroxy-phenylamino)-N-[2-(4-methyl-[1,4]diazepan-1-yl)-2-oxo-ethyl]-2-[2-(3,4,5-trihydroxy-6-hydroxymethyl-tetrahydro-pyran-2-yloxy)-phenyl]-acetamide

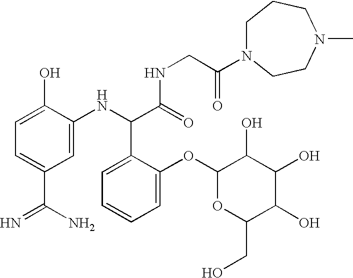

Preparation was carried out analogously to Example 1; 4-hydroxy-3-aminobenzamidine dihydrochloride and 2-isocyano-1-(4-methyl-[1,4]-diazepan-1-yl)-ethanone were used as starting materials. 1-Methyl-[1,4]diazepane was used as the amine component for the isonitrile.
C₂₉H₄₀N₆O₉ (616.68)
MS (ESI): 617.2 [M+H]

Synthesis of 3-amino-4-hydroxybenzamidine dihydrochloride: 3-Amino-4-hydroxybenzamidine dihydrochloride can be prepared in two steps from commercially available 4-hydroxy-3-nitro-benzonitrile. For the purpose, 3.3 g (20 mmol) of 4-hydroxy-3-nitro-benzonitrile are dissolved in 80 ml of dry chloroform and, whilst cooling with ice, 40 ml of a saturated solution of HCl in methanol are added. After 24 hours, the white suspension is filtered off and the imido ether obtained is dried. The latter is treated with 60 ml of a 2N solution of ammonia in methanol and is refluxed for 5 hours. After cooling, there is filtered off again a precipitate which is dissolved in 2N HCl solution in methanol and the solvent is then removed. The hydrochloride of 4-hydroxy-3-nitrobenzamidine obtained (3.3 g, yield 76%) is dissolved in 150 ml of methanol; 330 mg of palladium on activated carbon are added and stirring is carried out under an atmosphere of hydrogen gas at room temperature for 24 hours. After filtering off the catalyst, the solvent is removed and the residue obtained is again dissolved in a 2N solution of HCl in methanol. After drying, 2.5 g of the dihydrochloride of 3-amino-4-hydroxybenzamidine are obtained, which can be used directly for further syntheses.
C₇H₉N₃O (151.17)
MS (ESI): 152 [M+H]

### Example 4

### 2-(3-Carbamimidoyl-phenylamino)-N-[2-(4-methyl-[1,4]diazepan-1-yl)-2-oxo-ethyl]-2-[2-(3,4,5-trihydroxy-6-hydroxymethyl-tetrahydro-pyran-2-yloxy)-phenyl]-acetamide

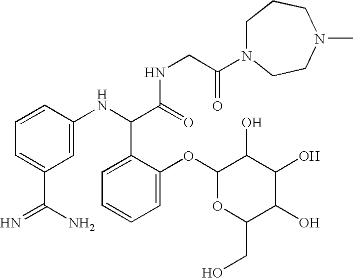

Preparation was carried out analogously to Example 1; 3-aminobenzamidine dihydrochloride and 2-isocyano-1-(4-methyl-[1,4]-diazepan-1-yl)-ethanone were used as starting materials. 1-Methyl-[1,4]diazepane was used as the amine component for the isonitrile.
C₂₉H₄₀N₆O₈ (600.68)
MS (ESI): 601.2 [M+H]

### Example 5

### 2-(5-Carbamimidoyl-2-hydroxy-phenylamino)-N-[2-(4-methyl-piperazin-1-yl)-2-oxo-ethyl]-2-[2-(3,4,5-trihydroxy-6-hydroxymethyl-tetrahydro-pyran-2-yloxy)-phenyl]-acetamide

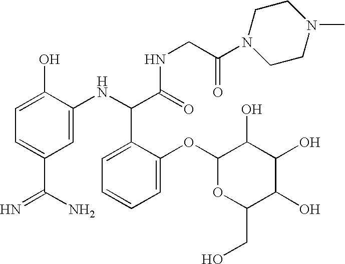

Preparation was carried out analogously to Example 1; 3-aminobenzamidine dihydrochloride and 2-isocyano-1-(4-methylpiperazin-1-yl)-ethanone were used as starting materials. N-Methylpiperazine was used as the amine component for the isonitrile.
C₂₈H₃₈N₆O₉ (602.65)
MS (ESI): 603 [M+H]

### Example 6

### 2-(5-Carbamimidoyl-2-hydroxy-phenylamino)-N-(2-oxo-2-thiomorpholin-4-yl-ethyl)-2-[2-(3,4,5-trihydroxy-6-hydroxymethyl-tetrahydro-pyran-2-yloxy)-phenyl]-acetamide

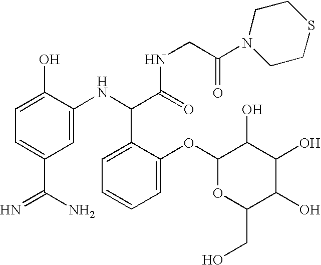

Preparation was carried out analogously to Example 1; 4-hydroxy-3-aminobenzamidine dihydrochloride and 2-isocyano-1-thiomorpholin-4-yl-ethanone were used as starting materials. Thiomorpholine was used as the amine component for the isonitrile.
C₂₇H₃₅N₅O₉S (605.67)
MS (ESI): 606 [M+H]

### Example 7

### 2-(3-Carbamimidoyl-phenylamino)-N-(2-oxo-2-thiomorpholin-4-yl-ethyl)-2-[2-(3,4,5-trihydroxy-6-hydroxymethyl-tetrahydro-pyran-2-yloxy)-phenyl]-acetamide

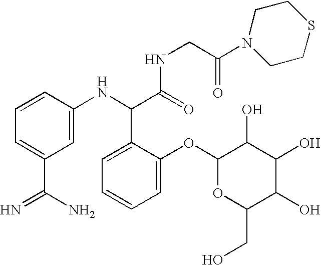

Preparation was carried out analogously to Example 1; 3-aminobenzamidine dihydrochloride and 2-isocyano-1-thio-morpholin-4-yl-ethanone were used as starting materials. Thiomorpholine was used as the amine component for the isonitrile.
C₂₇H₃₅N₅O₈S (589.67)
MS (ESI): 590 [M+H]

### Example 8

### 2-(5-Carbamimidoyl-2-hydroxy-phenylamino)-N-[2-(2-hydroxymethyl-piperidin-1-yl)-2-oxo-ethyl]-2-[2-(3,4,5-trihydroxy-6-hydroxymethyl-tetrahydro-pyran-2-yloxy)-phenyl]-acetamide

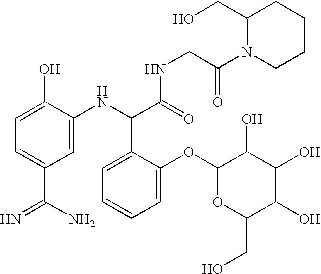

Preparation was carried out analogously to Example 1; 4-hydroxy-3-aminobenzamidine dihydrochloride and 1-(2-hydroxymethyl-piperidin-1-yl)-2-isocyano-ethanone were used as starting material. Piperidin-2-yl-methanol was used as the amine component for the isonitrile.
C₂₉H₃₉N₅O₁₀ (617.66)
MS (ESI): 618 [M+H]

### Example 9

### 2-(3-Carbamimidoyl-phenylamino)-N-[2-(2-hydroxymethyl-piperidin-1-yl)-2-oxo-ethyl]-2-[2-(3,4,5-trihydroxy-6-hydroxymethyl-tetrahydro-pyran-2-yloxy)-phenyl]-acetamide

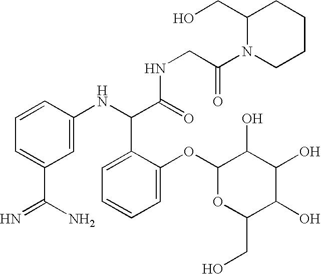

Preparation was carried out analogously to Example 1; 3-aminobenzamidine dihydrochloride and 1-(2-hydroxymethyl-piperidin-1-yl)-2-isocyano-ethanone were used as starting materials. Piperidin-2-yl-methanol was used as the amine component for the isonitrile.
C₂₉H₃₉N₅O₉ (601.66)
MS (ESI): 602 [M+H]

### Example 10

### 2-(5-Carbamimidoyl-2-hydroxy-phenylamino)-N-[2-(4-hydroxy-piperidin-1-yl)-2-oxo-ethyl]-2-[2-(3,4,5-trihydroxy-6-hydroxymethyl-tetrahydro-pyran-2-yloxy)-phenyl]-acetamide

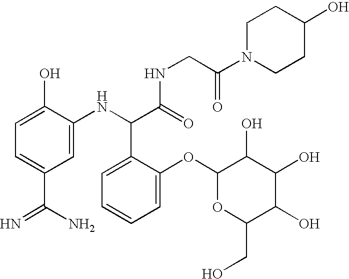

Preparation was carried out analogously to Example 1; 4-hydroxy-3-aminobenzamidine dihydrochloride and 1-(4-hydroxypiperidin-1-yl)-2-isocyano-ethanone were used as starting materials. Piperidin-4-ol was used as the amine component for the isonitrile.
C₂₈H₃₇N₅O₁₀ (603.63)
MS (ESI): 604 [M+H]

Determinations of the inhibitory action towards factor Xa activity were carried out in accordance with the methods described in WO0216312.

The IC₅₀ value of the compound given in Example 1 is in the range from 0.1 nM to 10 nM. The IC₅₀ values of the compounds given in Examples 2 to 10 are in the range from 0.1 nM to 1 μM.

1-10. (canceled)

11. A compound of formula (I): 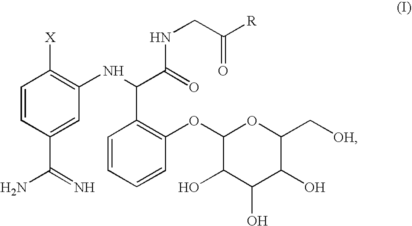
wherein
X is a hydrogen atom,a hydroxy group, a C₁—, C₂—, C₃— or C₄-alkyloxy group or a fluorine atom and
R is an optionally substituted heterocycloalkyl radical having 5, 6 or 7 ring atoms,
or a pharmacologically acceptable salt, solvate, hydrate or pharmacologically acceptable formulation thereof.

12. A compound of claim 11 wherein X is a hydrogen atom or a hydroxy group.

13. A compound of claim 11 wherein R is a group of formula 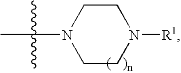
wherein n is 0, 1 or 2 and R¹ is a C₁—, C₂—, C₃— or C₄-alkyl group.

14. A compound of claim 11 wherein R is a cyclic group of formula —N(CH₂CH₂)₂NCH₃.

15. A compound of claim 11 wherein the stereochemistry of the 3,4,5-trihydroxy-6-hydroxymethyl-tetrahydropyran-2-yloxy group corresponds to that of β-D-glucose.

16. A compound of claim 11 wherein the compound has an (S) configuration at the phenylglycine entity.

17. A pharmaceutical composition comprising a compound of claim 11.

18. A pharmaceutical composition of claim 17 further comprising a carrier substance and/or adjuvant.

19. A method for inhibiting factor Xa comprising administering a compound of claim 11 to a subject.

20. The method of claim 19 wherein the subject is in need of factor Xa inhibition.

21. The method of claim 19 wherein the subject has been identified as in need of factor Xa inhibition and the compound is administered to the identified subject.

22. A method of treating a subject suffering from or susceptible to a thromboembolic condition, arterial restenosis, septicaemia, cancer, or acute inflammation, comprising administering to the subject a compound of claim 11.

23. The method of claim 22 wherein (i) the subject is identified as suffering from or susceptible to a thromboembolic condition, arterial restenosis, septicaemia, cancer, or acute inflammation and (ii) the compound is administered to the identified subject.

24. The method of claim 22 wherein (i) the subject is identified as suffering from a thromboembolic condition, arterial restenosis, septicaemia, cancer, or acute inflammation and (ii) the compound is administered to the identified subject.

25. A method of treating a subject suffering from or susceptible to a condition mediated by factor Xa activity, comprising administering to the subject a compound of claim 11.

26. The method of claim 25 wherein the subject is identified as suffering from or susceptible to a condition mediated by factor Xa activity and the compound is administered to the identified subject.

27. A method of treating a subject undergoing vascular surgery, comprising administering to the subject a compound of claim 11.
